(19) United States
(12) Patent Application Publication
(10) Pub. No.: US 2009/0188815 A1
(43) Pub. Date: Jul. 30, 2009

(54) Transfer dish for dental materials

(75) Inventor: Marcus Oliver Ahlers, Hamburg (DE)

Correspondence Address: MORRISON & FOERSTER LLP, 1650 TYSONS BOULEVARD, SUITE 400, MCLEAN, VA, 22102, US

(73) Assignee: Ernst Muhlbauer Gmbh & Co. KG, Norderfriedrichskoog (DE)

(21) Appl. No.: 11/664,390

(22) PCT Filed: Sep. 30, 2005
(86) PCT No.: PCT/EP2005/010605

§ 371 Date: Mar. 30, 2007

(30) Foreign Application Priority Data
Oct. 1, 2004 (DE) 20 2004 015 300.9

(51) Int. Cl. A61B 19/02; A61C 19/00

(52) U.S. Cl. 206/63.5; 433/25

(57) ABSTRACT

A transfer dish for dental materials has a bottom surface, a top surface and a front surface. The top surface is designed with troughs for receiving pre-dosed amounts of dental material for application which have, in the front area, a plane ramp sloping toward a rear area, and, in the rear area, the troughs have a concave wall adjacent to a transition area. The transition area is designed as an osculating curve of the concave wall with respect to the plane ramp, and the trough, in a top view, has the contour of a U which is open toward a front area of the transfer dish. The ramp tapers in width toward the front area of the transfer dish.

### REFERENCE TO RELATED APPLICATIONS

This application is the national stage under 35 USC 371 of International Application No. PCT/EP2005/010605, filed Sep. 30, 2005, which claims the Paris Convention priority of German Application No. 20 2004 015 300.9, filed Oct. 1, 2004, the entire contents of which are incorporated herein by reference.

### FIELD OF THE INVENTION

The invention relates to a transfer dish for dental materials, with a bottom surface, a top surface, and a front surface directed toward the user, the top surface being designed with troughs for receiving pre-dosed amounts of dental material for application.

### BACKGROUND OF THE INVENTION

When treating teeth, dentists in many cases require dental materials that range from liquid to kneadable materials. These dental materials are generally prepared by assistants, placed on a transfer dish and passed to the dentist who then applies them. The dental materials can, for example, be materials for preparing fillings. In their initial state, these materials can be shaped in order to fit them into or place them onto the tooth, where they are then cured by means of a chemical reaction. Since the time during which these shapeable dental materials can be worked is limited, the dental assistants usually place them in pre-dosed amounts onto the transfer dish just shortly before their application. To apply the dental material, the dentist removes it using a suitable instrument, for example a Heidemann spatula for modeling of fillings. To reduce the risk of loss of material as a result of curing taking place too early, the dental material is in each case pre-dosed or pre-mixed in small portions. When using materials that are cured by the action of light, a situation where curing takes place too early can be avoided by providing the transfer dish with a lid that is not transparent to light. This lid is impervious to light, at least for the wavelengths that lead to the curing of the dental material. To ensure that the lid only needs to be opened by the dentist immediately before use of the dental material, it must also be easy to open during the treatment. In transfer dishes known from prior public use, difficulties arise in this respect, with the result that a second helping hand is often needed.

Transfer dishes known from prior public use involve handling difficulties in terms of the aforementioned aspects. Removing the pre-dosed dental material from the troughs using a suitable tool, in particular the Heidemann spatula, often proves difficult. In addition, with a lid that is difficult to open, accessing the dental materials is often only possible with assistance.

### SUMMARY OF THE INVENTION

The object of the invention is to improve a transfer dish of the type mentioned at the outset, in such a way that the abovementioned disadvantages are avoided or at least minimized.

The solution according to the invention lies in the features of the invention as broadly described herein. Advantageous developments form the subject matter of the preferred embodiments.

According to the invention, a transfer dish for dental materials, with a bottom surface, top surface and front surface, the top surface being designed with troughs for receiving pre-dosed amounts of dental material for application, wherein, in the front area, the troughs have a plane ramp sloping toward the rear, and in the rear area they have a concave wall adjacent to a transition area.

With the design according to the invention, a transfer dish is made available in which the troughs have a shape that is favorable in terms of handling. The core aspect of the invention lies in the concept of designing the trough as a combination of a plane surface (ramp) sloping downward in the direction away from the user (rearward) and of an adjoining concave, preferably semicircular wall. This results in a smooth trough that is not interrupted by any steps or edges and that is suitable for safe storage and transporting of the dental material but also permits simple removal of the dental material via the ramp. The ramp according to the invention, with its plane surface, forms a suitable base on which the dental material can be pre-modeled and removed with the tool used for application, such as the Heidemann spatula. Unlike a curved surface, the planar nature of the ramp permits optimal contact with the tool, such that the dental material can be removed in a controlled manner. Handling difficulties caused by a spatula that does not lie flat are thus effectively avoided. With its plane surface, the ramp also allows the pre-portioned dental material in the trough to be distributed into subsidiary portions and thus processed further in smaller amounts, for example mixed with a further component. Another advantage of the invention lies in the fact that the plane surface of the ramp makes available an additional working surface inside the trough. The handling of the dental material is thus made considerably easier.

The transition area between the ramp and the concave wall is preferably rounded. This permits a particularly favorable transition between the rear area and the front area. The tool can be guided between the front area and the rear area without having to overcome an edge. This further improves handling and removability. In this connection, it is particularly preferred if the transition area to the plane surface of the ramp is designed as an osculating curve of the concave wall. The concave wall adjoins the plane surface of the ramp tangentially. This results in a particularly smooth transition which guarantees optimal removability of the dental material. It will be appreciated that the radius of curvature of the concave wall in this area is chosen to be as great as possible, such that only small bend angles result. The radius of curvature of the wall in the parts more remote from the transition area can of course be smaller.

The trough is expediently shaped such that, in a top view, it has the contour of a letter U which is open toward the front. By means of this U-shaped configuration, the ramp acquires the form of a channel, such that a certain guiding of the tool can be achieved. The risk of uncontrolled distribution of the dental material beyond the area of the trough is thus minimized. The U-shape is preferably such that the clear width of the ramp tapers toward the front. This means that the channel created by the ramp narrows toward the front. A funnel effect can thus be achieved, by which even small residual amounts of dental material remaining in the trough can be pushed forward, so that it can then be removed together. This considerably improves handling even with small amounts of dental material.

It may be expedient for the ramp to be provided with a step in its front half. In this way, a scraper edge is made available with which the tool can be freed of excess dental material.

The top surface is preferably designed such that it slopes upward toward the rear (in the direction away from the user). This results in a kind of desk shape which, even with several troughs arranged offset or in rows on the top surface, ensures easy removability of material from all the troughs.

The transfer dish, at least in the area of the troughs, is expediently made of a material that has the greatest possible scratch resistance. This ensures that, even after prolonged use, the transfer dish retains a high surface quality, despite its being subjected to the action of the tool for removing the dental material.

According to a further aspect of the invention that may merit independent protection, the transfer dish has a multi-part design with a housing and a pallet-like trough insert. This means that the housing and pallet can be made of different materials and can thus be adapted to their respective function. It also makes it possible to provide different trough inserts, which can be fitted into the housing depending on requirements.

The housing is preferably made of metal. Metal or another material with a high specific density has the advantage that the housing is stable because of its weight, such that it does not inadvertently slip out of place under the force of the tool used to remove the dental material. The transfer dish does not then need to be held securely by a second hand, and instead it can be handled with just one hand. The term metal is to be interpreted widely here and also includes other suitable materials with a high specific density.

The trough insert is preferably designed to be exchangeable and is made of a material that can be autoclaved. This permits simple hygienic cleaning of the trough insert coming into contact with the dental material.

According to another preferred embodiment, which may merit independent protection, a lid is provided which, in the closed state, covers the troughs and which is arranged on the transfer dish via an over-center helper mechanism. The lid is preferably made of a material that is not transparent to light and ensures that the dental material introduced into the troughs does not cure too early. By virtue of the over-center helper mechanism, the lid is stable in two positions, that is to say it automatically stays in place both in the closed position and also in the opened position. The opened position is preferably chosen such that the lid does not lie flat on the supporting surface but instead extends upward transverse to said surface. This means it can be easily gripped and closed again when necessary. There is no longer any need for separate locking devices for the closed position and the opened position. Thus, the arrangement of the lid according to the invention also allows said lid to be folded open and closed with just one hand.

The over-center helper mechanism expediently comprises a pivot hinge arranged below the top surface. This provides a structurally simple possibility by which the lid with an over-center helper mechanism can be attached to the transfer dish. The lid is expediently L-shaped in cross section and is articulated at the far end of its short branch.

The bottom surface is preferably provided with feet that have an anti-slip coating. This increases the stability with which the transfer dish stands on the supporting surface. In particular, this prevents the transfer dish from slipping away during opening and closing of the lid and during handling of the dental material. This further increases the ability of the transfer dish to be maneuvered with just one hand.

### BRIEF DESCRIPTION OF THE DRAWINGS

The invention is explained in more detail below on the basis of an illustrative embodiment and with reference to the attached drawing, in which:

FIG. 1 shows a perspective overall view of an illustrative embodiment of a transfer dish according to the invention;

FIG. 2 shows a side view, in cross section, of the transfer dish depicted in FIG. 1;

FIG. 3 shows an enlarged partial view of the transfer dish depicted in FIG. 1; and

FIG. 4 shows a detail of the cross section depicted in FIG. 2.

### DETAILED DESCRIPTION OF THE INVENTION

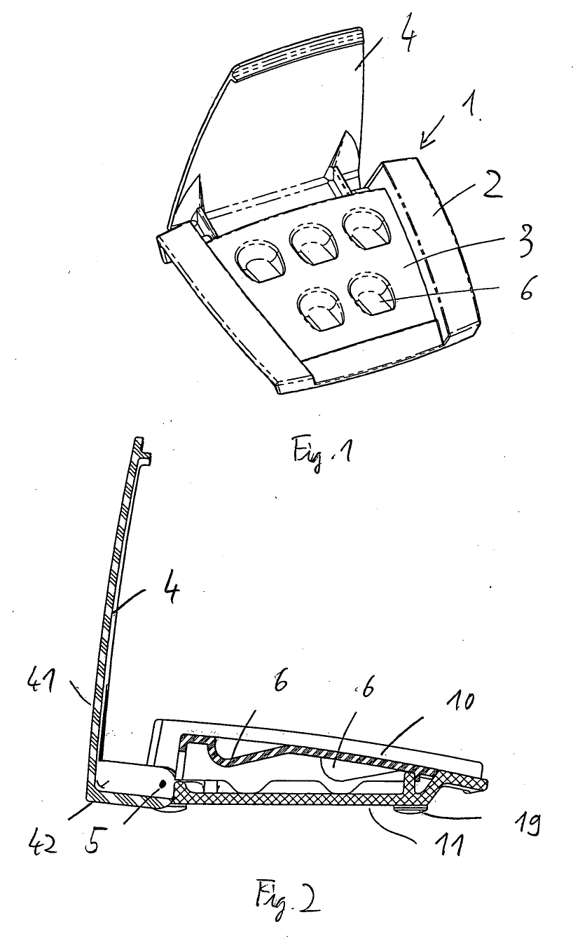

An illustrative embodiment of a transfer dish according to the invention is shown in FIGS. 1 to 4 in the drawing. It is designated in its entirety by reference number 1. The transfer dish 1 according to the invention is composed of three main components, namely a housing 2, a pallet insert 3, and a lid 4.

The housing 2 is made from a metal material of high density, for example diecast zinc. It has, when seen in elevation, a trapezoidal shape, wherein its front face directed toward the user has a lesser height than its rear face directed away from the user. On its bottom surface 11, the housing 2 is provided with a plurality of feet 19. Each foot 19 is located at a corner of the trapezoidal base layout of the housing 2. The bottom surface of each foot 19 is provided with a rubber coating which ensures a high coefficient of friction in relation to a supporting surface (not shown) on which the transfer dish 1 according to the invention is placed. The supporting surface in question is preferably a swivel tray of the kind that is found on a patient's chair used by dentists. The rubber coating of the feet 19 is preferably adapted to provide a good hold on the surface of the swivel tray and thus to ensure a high degree of safety against accidental displacement. Provided on the top surface 10 of the housing 2, and extending across most of the width of the housing 2, there is a depression into which the pallet insert 3 is fitted. The pallet insert 3 is essentially plane and is oriented parallel to the inclination of the top surface 10 of the housing 2. The pallet insert 3 comprises a plurality of troughs 6 arranged in rows (two rows in the illustrative embodiment shown). The troughs 6 can have a numbering or inscription arranged on the pallet insert 3. They are formed integrally from the material of the pallet insert 3. The material used for the pallet insert 3 is preferably a polyamide, for example Grilamid® from EMS-Chemie AG. The latter has the advantage that it can be cleaned in an autoclave, and it also has the advantage that it is very resistant to scratching.

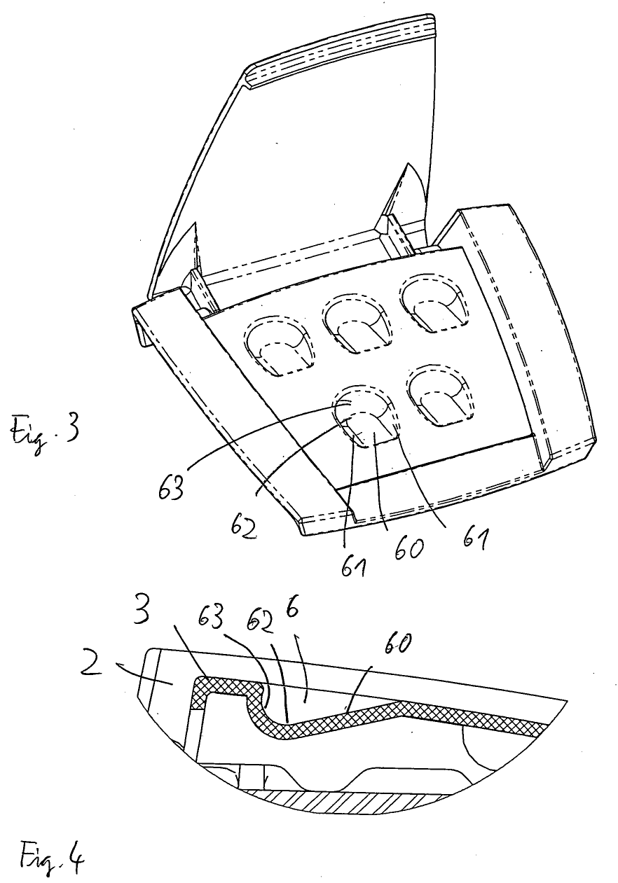

The troughs 6 are divided into areas. In the front area, a plane surface 60 is formed which extends like a ramp sloping downward from the front edge of the trough toward the rear. The ramp 60 extends across almost the complete width of the trough 6 and has side walls 61 located on both sides thereof. The transition from the side walls 61 to the plane ramp 60 is preferably rounded, while the transition from the side wall 61 to the top surface of the pallet insert 3 can also be angular. A concave wall 63 is arranged in the rear area of the trough 6. It extends, as can be seen in particular from FIG. 3, in a semicircle formation round the rear end of the ramp 60 and connects the latter's two side walls 61 to one another. A transition area 62 between the ramp 60 and the concave wall 63 is designed such that the obtained transition is free of any steps or edges. As can be seen in FIG. 4, this transition is expediently configured such that, in the area of the transition 62, the plane of the ramp 60 forms a tangent to the concave wall 63. A particularly favorable transition is obtained with an osculating curve.

As can further be seen from FIG. 3, the trough 6, seen in a top view, has the shape of a letter U which is open toward the front. It is expediently configured such that the legs of the U are closer together at their free end, which results in a formation tapering toward the front. This facilitates the collection and removal of dental material arranged in the trough 6, particularly by means of a tool such as a Heidemann spatula.

The lid 4 is made of a plastic, for example a polyamide, e.g. Grilamid® from EMS-Chemie AG. The nature of the plastic is such that the lid 4 is non-transparent to light in the wavelength range at which the dental material received in the troughs 6 cures. The lid 4 is of an L-shaped design in cross section, with a longer branch 41 and a shorter branch 42. The dimensions of the longer branch 41 in terms of length and width are chosen such that the pallet insert 3 is covered when the lid 4 is in the closed state. At its outer end, the shorter branch 42 is connected to the housing 2 in an articulated manner via a pivot hinge 5. The length of the shorter branch 42 is chosen to match the height of the housing 2 in the rear area in such a way that the lid 4, in the closed state, ends flush with the top surface 10 of the housing 2, and, in the opened state, the shorter branch 42 comes to lie approximately horizontal (deviation ±10 degrees) on the supporting surface. Instead of the placement on the supporting surface, a stop can also be provided that holds the lid 4 in the opened position with the long branch 41 projecting upward. The L-shaped configuration of the lid 4 and the arrangement of the pivot hinge 5 results in an over-center helper mechanism which keeps the lid stable in two positions, namely in its closed state, resting on the pallet insert 3, and also in its opened state, projecting upward behind the housing 2. Separate fixing means for the respective positions are therefore not needed.

1. A transfer dish for dental materials, having a bottom surface, a top surface and a front surface,
the top surface being designed with troughs for receiving pre-dosed amounts of dental material for application which have, in the front area, a plane ramp sloping toward a rear area, and, in the rear area, the troughs have a concave wall adjacent to a transition area.

2. The transfer dish as claimed in claim 1, wherein the transition area is designed as an osculating curve of the concave wall with respect to the plane ramp.

3. The transfer dish as claimed in claim 1 or 2, wherein the trough, in a top view, has the contour of a U which is open toward a front area of the transfer dish.

4. The transfer dish as claimed in claim 3, wherein the ramp tapers in width toward the front area of the transfer dish.

5. The transfer dish as claimed in claim 1 or 2, wherein the ramp has a step in its front half.

6. The transfer dish as claimed in claim 1 or 2, the further comprising a housing designed with a top surface that slopes upward toward the rear of the transfer dish.

7. The transfer dish as claimed in claim 1 or 2, wherein the transfer dish is designed to be scratch-resistant in the area of the troughs.

8. The transfer dish as claimed in claim 1 or 2, wherein the dish has a multi-part design with a housing and a pallet insert.

9. The transfer dish as claimed in claim 8, wherein the housing is made of metal.

10. The transfer dish as claimed in claim 8, wherein the pallet insert is designed to be exchangeable and can be autoclaved.

11. The transfer dish as claimed in claim 1 or 2, wherein the lid covers the troughs when in a closed state and is arranged on the dish by way of an over-center helper mechanism.

12. The transfer dish as claimed in claim 11, wherein the over-center helper mechanism has a pivot hinge arranged below the top surface.

13. The transfer dish as claimed in claim 11, wherein the lid is L-shaped in cross section and is articulated at the far end of its short branch.

14. The transfer dish as claimed in claim 1 or 2, wherein the bottom surface is provided with feet that have an anti-slip coating.

15. The transfer dish as claimed in claim 4, wherein the ramp has a step in its front half.

16. The transfer dish as claimed in claim 4, further comprising a housing designed with a top surface that slopes upward toward the rear of the transfer dish.

17. The transfer dish as claimed in claim 6, wherein the dish has a multi-part design with a housing and a pallet insert.

18. The transfer dish as claimed in claim 17, wherein the housing is made of metal.
